(19) United States
(12) Patent Application Publication
(10) Pub. No.: US 2008/0199449 A1
(43) Pub. Date: Aug. 21, 2008

(54) Methods and Compositions for Use in Treating Vascular Diseases and Conditions

(75) Inventors: Randall T. Peterson, Belmont, MA (US); Charles C. Hong, Nolensville, TN (US)

Correspondence Address: CLARK & ELBING LLP, 101 FEDERAL STREET, BOSTON, MA, 02110, US

(73) Assignee: The General Hospital Corporation, Boston, MA (US)

(21) Appl. No.: 12/063,545

(22) PCT Filed: Aug. 11, 2006
(86) PCT No.: PCT/US06/31375

§ 371 Date: Feb. 11, 2008

Related U.S. Application Data
(60) Provisional application No. 60/707,613, filed on Aug. 12, 2005.

(51) Int. Cl. A61K 31/343; A61K 38/02; A61K 31/352; A61K 31/5365; A61K 31/7088; A61K 38/45; A61K 38/16; A61P 9/10

(52) U.S. Cl. 424/94.5; 514/468; 514/2; 514/456; 514/233.5; 514/44; 514/12

(57) ABSTRACT

The invention includes methods and compositions for use in treating or preventing vascular disease or promoting vascular growth or development. The methods and compositions can be used, for example, in the treatment of diseases associated with ischemia, such as heart attack and stroke.

### BACKGROUND OF THE INVENTION

This invention relates to methods of treating or preventing vascular disease or promoting vascular growth or development.

Angiogenesis and vasculogenesis are processes involved in the growth of blood vessels. Angiogenesis is the process by which new blood vessels are formed from extant capillaries, while vasculogenesis involves the growth of vessels from endothelial progenitor cells. Angiogenesis and vasculogenesis, and the factors that regulate these processes, are important in embryonic development, inflammation, and wound healing, and also contribute to pathologic conditions such as tumor growth, rheumatoid arthritis, and chronic inflammatory diseases (see, e.g., U.S. Pat. No. 5,318,957; Yancopoulos et al., Cell 93:661-664, 1998; Folkman et al., Cell 87:1153-1155, 1996; and Hanahan et al., Cell 86:353-364, 1996).

Both angiogenesis and vasculogenesis involve the proliferation of endothelial cells that line the walls of blood vessels. The angiogenic process involves not only increased endothelial cell proliferation, but also includes a cascade of additional events, including protease secretion by endothelial cells, degradation of the basement membrane, migration through the surrounding matrix, proliferation, alignment, differentiation into tube-like structures, and synthesis of a new basement membrane. Vasculogenesis involves recruitment and differentiation of mesenchymal cells into angioblasts, which then differentiate into endothelial cells that form de novo vessels (see, e.g., Folkman et al., Cell 87:1153-1155, 1996).

Several angiogenic and/or vasculogenic agents with different properties and mechanisms of action are known in the art. For example, acidic and basic fibroblast growth factors (aFGF and bFGF), transforming growth factors alpha and beta (TGF-α and TGF-β), tumor necrosis factor (TNF), platelet-derived growth factor (PDGF), vascular endothelial cell growth factor (VEGF), and angiogenin are potent and well-characterized angiogenesis-promoting agents. In addition, both nitric oxide and prostaglandin have been shown to be mediators of various angiogenic factors, such as VEGF and bFGF.

Cell signaling via 3′-phosphorylated phosphoinositides has been implicated in a variety of cellular processes, including malignant transformation, growth factor signaling, inflammation, and immunity (see Rameh et al., J. Biol. Chem. 274:8347-8350, 1999, for a review). The enzyme responsible for generating these phosphorylated signaling products, phosphatidylinositol 3-kinase (PI 3-kinase; PI3K), was originally identified as an activity associated with viral oncoproteins and growth factor receptor tyrosine kinases that phosphorylates phosphatidylinositol (PI) at the 3′-hydroxyl of the inositol ring (Panayotou et al., Trends Cell. Biol. 2:358-360, 1992). Specific inhibitors of PI3K, LY294002 and wortmannin, have been reported to be useful for preventing angiogenesis in tumors (see, for example, Su et al., Cancer Res. 63:3585, 2003).

Angiogenesis and vasculogenesis have been the focus of intense interest, as these processes can be exploited to therapeutic advantage. Stimulation of angiogenesis and/or vasculogenesis can aid in, for example, the healing of wounds, the vascularizing of grafts (e.g., skin grafts), and the enhancement of collateral circulation (e.g., in cases of vascular occlusion or stenosis). Stimulation of these processes can also be beneficial in treating or preventing ischemia, which occurs when a tissue does not receive an adequate supply of oxygen. Approaches to stimulate angiogenesis and/or vasculogenesis can involve the use or manipulation of the agents listed above, such as VEGF.

gridlock (grl) is an artery-restricted gene that has been characterized in the zebrafish model and other systems (Peterson et al., Nat. Biotech. 22 (5):595-599, 2004). This gene is expressed in the lateral posterior mesoderm and guides the arterial-venous fate decision. Graded reduction of grl expression, by mutation or morpholino antisense oligonucleotides, progressively ablates regions of the artery and expands contiguous regions of the vein, preceded by an increase in expression of the venous markers and diminution of the expression of arterial markers. Zebrafish homozygous for a grl mutation exhibit a morphological defect of the dorsal aorta that prevents circulation to the trunk and tail, while circulation to the head is maintained. This phenotype is similar to the human congenital disorder aortic coarctation, a condition that affects nearly 1 in 1,000 live births and is a major source of morbidity and mortality in those affected. In mammalian cells, the gridlock gene has been shown to play roles in vasculogenesis, including the progression from endothelial cell proliferation and migration to vascular network formation. Mice lacking the gridlock gene exhibit ventricular septal defects, cardiomyopathies, and vascular defects.

A better understanding of the underlying mechanisms associated with angiogenesis, vasculogenesis, and arterial differentiation are needed to identify new methods of treating vascular diseases.

### SUMMARY OF THE INVENTION

The invention features methods of treating or preventing vascular diseases or promoting vascular (arterial or venous) growth or development. In the case of promoting arterial growth, the methods can involve administering to a patient an agent that activates p44 Mitogen-Activated Protein (MAP) kinase/Extracellular signal-Regulated Kinase (ERK) (hereinafter referred to as “ERK”) in an amount sufficient to achieve the desired treatment or prophylaxis, or to promote the growth or development. In one example, ERK is activated by administering an agent that inhibits PI3K and/or AKT.

The invention also provides methods of inducing angioblasts to undergo arterial differentiation by contacting the angioblasts with an effective amount of an agent that activates ERK (e.g., an agent that inhibits PI3K and/or AKT).

In any of the above-described methods, the PI3K inhibitor can be, for example, a small molecule, a peptide, a protein (e.g., PLC-γ, PKC, Raf, MEK, or ERK), or a nucleic acid molecule (e.g., a nucleic acid molecule encoding PLC-γ, PKC, Raf, MEK, or ERK). Small molecule inhibitors of PI3K that can be used in the methods of the invention include, without limitation, wortmannin, LY294002, and compounds of formula (I):

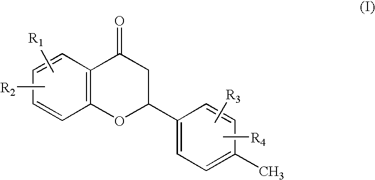

In formula (I), each of R₁, R₂, R₃, and R₄ are, independently, selected from H, halide, CF₃, C₁₋₃ alkyl (unsubstituted or substituted), C₁₋₃ alkoxy, OH, SH, NO₂, and CN. For example, the compound of formula (I) can be GS4898, as shown below.

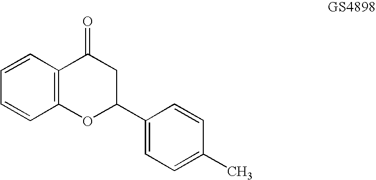

Peptide inhibitors of PI3K that can be used in the methods of the invention include, without limitation, SH2 domain binding peptides, such as PI3K-SH2-OMT.

In one example of a method of the invention, a patient who has or is at risk of developing a disease or condition of the aorta, such as congenital dysplasia of the aorta (e.g., coarctation of the aorta) is treated using the compounds described herein. In another example, the vascular disease to be treated or prevented is ischemia. Examples of tissues in which ischemia can occur, and which can be treated according to the invention, include, without limitation, muscle (e.g., cardiac muscle), brain, kidney, and lung. Ischemic diseases that can be treated or prevented using the methods of the invention include, for example, cerebrovascular ischemia, renal ischemia, pulmonary ischemia, limb ischemia, ischemic cardiomyopathy, and myocardial ischemia. The ischemia can result from, for example, a wound, surgery, vascular occlusion, or vascular stenosis.

The methods described herein can also be used to treat patients suffering or at risk of suffering a heart attack, stroke, or peripheral vascular disease. Further, the methods can be used for enhancing angiogenesis to accelerate wound healing, the vascularization of surgically transplanted tissue, or the healing of a surgically-created anastomosis.

The methods described herein can also be used to promote arterial vascular growth or development of an immature vessel. For example, immature vessels can be found in the diseased eyes of patients suffering from diabetic retinopathy or exudative (e.g., wet) age related macular degeneration. Using the methods described herein, agents (e.g., agents that activate ERK (e.g., PI3K and/or AKT inhibitors) or increase ERK levels) can be administered for the treatment of diabetic retinopathy and exudative age related macular degeneration.

The invention also includes pharmaceutical compositions that include one or more compounds of formula (I) and a pharmaceutically acceptable excipient. For example, the compositions can include GS4898. Optionally, the compositions can also include one or more additional therapeutic agents (e.g., an agent that activates and/or increases ERK levels (e.g., another PI3K inhibitor)), such as those described herein.

The invention also includes methods of promoting venous vascular growth or development in patients. These methods can involve administering to patients an agent that inactivates or decreases the levels of p44 Mitogen-Activated Protein (MAP) kinase/Extracellular signal-Regulated Kinase (ERK) or a component of the ERK pathway in an amount sufficient to promote the venous growth or development. Further, the invention includes methods of inducing angioblasts to undergo venous differentiation. These methods can involve contacting the angioblasts with an effective amount of an agent that deactivates or decreases levels of p44 Mitogen-Activated Protein (MAP) kinase/Extracellular signal-Regulated Kinase (ERK) or a component of the ERK pathway.

In addition to the therapeutic methods described herein, the invention also includes the use of the agents described herein for the prevention and treatment of diseases and conditions such as those described herein, as well as the use of such agents in the preparation of medicaments for these purposes.

In the generic descriptions of the compounds used in the invention, the number of atoms of a particular type in a substituent group is generally given as a range, e.g., an alkyl group containing from 1 to 3 carbon atoms or C₁₋₃ alkyl. Reference to such a range is intended to include specific references to groups having each of the integer number of atoms within the specified range. For example, an alkyl group from 1 to 3 carbon atoms includes each of C₁, C₂, and C₃. Other numbers of atoms and other types of atoms can be indicated in a similar manner.

As used herein, the term “alkyl” and the prefix “alk-” are inclusive of both straight chain and branched chain groups and of cyclic groups. C₁₋₃ alkyl groups may be substituted or unsubstituted. Exemplary substituents include halide, hydroxyl, fluoroalkyl, perfluoralkyl, amino, aminoalkyl, disubstituted amino, and carboxyl groups. C₁₋₃ alkyls include, without limitation, methyl, ethyl, n-propyl, isopropyl, and cyclopropyl.

By “halide” is meant bromine, chlorine, iodine, or fluorine.

By “alkoxy” is meant a chemical substituent of the formula —OR, where R is selected from C₁₋₃ alkyl.

As used herein, a “pharmaceutical composition” refers to a formulation of a compound (e.g., a compound of formula (I)) that would be suitable for administration to humans or other animals. For example, such compositions can be in a form that is suitable for approval by the Food and Drug Administration (FDA). The pharmaceutical compositions could be suitable for approval if the compositions meet efficacy and toxicity standards established by the FDA. The pharmaceutical compositions can be shown to meet these standards using established methods of testing that are acceptable to the FDA. To reduce the toxicity of the pharmaceutical compositions, each of the components of the compositions is required to meet standards of purity. Thus, for example, compounds of formula (I) can be purified to remove reaction side-products (e.g., any product formed during the synthesis of a compound of formula (I) that is not the desired compound) and reaction residues (e.g., reaction solvents, reagents, and salts) prior to the formulation of the pharmaceutical compositions. For the pharmaceutical compositions described herein, the reaction side-products and residues are preferably less than 2%, 1%, 0.5%, or 0.1% (w/w) of the mass of the compound of formula (I) used in the pharmaceutical formulation.

As used herein, the term “treating” refers to administering a pharmaceutical composition for prophylactic and/or therapeutic purposes. To “prevent” disease refers to prophylactic treatment of a human patient who is not yet ill, but who is susceptible to, or otherwise at risk of, a particular disease. To “treat” disease or use for “therapeutic treatment” refers to administering treatment to a patient already suffering from a disease to improve or stabilize the patient's condition. Thus, as used herein, “treating” is the administration to a human patient either for therapeutic or prophylactic purposes.

As used herein, the terms “administration” and “administering” refer to methods of giving a dosage of a pharmaceutical composition to a patient, where the methods are, e.g., intramuscular, topical, oral, intravenous, intraperitoneal, or subcutaneous (also see below). The method of administration selected can vary depending on various factors, e.g., the components of the pharmaceutical composition, the site of the potential or actual disease, and the severity of disease.

As used herein, an “amount sufficient” is an amount of an agent (e.g., an ERK protein activator and/or a PI3K inhibitor) that, in vivo, provides a desired effect (e.g., promotes arterial vascular growth or development, or ameliorates one or more of the symptoms of a disease condition mentioned herein). This amount can vary depending on various factors, e.g., the agent being used, the site of the potential or actual disease, the severity of disease, the route of administration, and the size of the patient. For any given combination of factors, a sufficient amount can be determined by using animal studies (e.g., the studies described herein) and standard clinical trials. By “effective amount” is meant an amount of an agent (e.g., an ERK protein activator and/or a PI3K inhibitor) that, in vitro or ex vivo, provides a desired effect (e.g., induces arterial differentiation of vessel tissue or angioblasts).

As used herein, “PI3K inhibitor” refers to an agent capable of reducing the enzymatic activity of PI3K by, for example, direct interaction with this protein under physiological conditions. Methods of assaying for PI3K inhibition activity are well known in the art. In general, the assays involve comparing the activity of PI3K (e.g. synthetic, recombinantly expressed, or purified PI3K) in the presence and absence of the candidate PI3K inhibitor. PI3K activity can be measured, for example, as described by Matter et al., Biochem. Biophys. Res. Comm. 186:624-631, 1992. Similarly, an “ERK activator” is an agent capable of activating the activity of ERK, as described herein.

The invention provides several advantages. As is discussed above, promoting blood vessel growth can be beneficial in the treatment of many diseases and conditions, including, for example, ischemia-related diseases and conditions. The prevalence of such conditions, including stroke, heart attack, limb ischemia, and others, is great, and these conditions are significant bases for patient mortality and morbidity, as well as substantial economic burdens. Thus, approaches to treating these diseases and conditions, such as the methods described herein, provide substantial benefits.

Other features and advantages of the invention will be apparent from the detailed description of the invention, the drawings, and the claims.

### BRIEF DESCRIPTION OF THE DRAWINGS

FIG. 1. Suppression of the gridlock phenotype by the novel flavone GS4898 (2-(4-methylphenyl)-4H-chromen-4-one) involves PI3 kinase inhibition. A, Suppression of the gridlock phenotype (atresia of the proximal aorta in grl^{m145} mutants) by GS4898. Above, microangiogram of an untreated 60-hpf grl^{m145} mutant, in which arterial circulation to the tail is disrupted. Below, microangiogram showing the restoration of tail circulation in a grl^{m145} mutant by GS4898 treatment. B, The gridlock phenotype is suppressed by GS4012, GS4898, LY294002 and wortmannin, but not PD98059 and quercetin. GS4898 and the PI3-K inhibitors are enclosed in dashed boxes. C, Quantitative Western analysis of activating AKT phosphorylation (at Ser472) in 20 somite-stage (ss) zebrafish embryos shows dramatic reduction in AKT phosphorylation by GS4898 (25 μM) or wortmannin (0.5 μM) treatment, starting at 10-hpf. Results normalized from 3 independent experiments for each condition. Error bars, standard error. P<0.004 for each, versus untreated. D, Dose response for GS4898, LY294002, and wortmannin. Compounds were added at 12-hpf and washed out at 27-hpf. Suppression (%) represents percentage of treated embryos that have tail circulation at 48-hpf. Number on top of each bar represents actual number of embryos with normal tail flow over the total number treated. E, Model for two VEGF receptor-dependent signaling branches with opposing effects on arterial specification. The PLC-γ/ERK branch mediates arterial cell specification, whereas the PI3-K branch exerts a negative effect on the PLC-γ/ERK branch, possibly via direct inhibition of Raf by Akt. By blocking PI3-K, GS4898 lifts PI3-K's inhibition of the PLC-γ/ERK pathway, leading to increased ERK activation and arterial specification. F, Synergy between GS4898 or PI3-K inhibitors and GS4012. Single treatment involving a sub-effective dose of GS4898 (0.75 μM), LY294002 (3 μM), wortmannin (0.075 μM), or GS4012 (0.7 μg/mL) does not suppress the gridlock phenotype, but co-treatment with GS4012 and either GS4898, LY294002, or wortmannin at the same doses leads to significant suppression of the gridlock phenotype.

FIG. 2. Diphosphorylated ERK is a specific marker of early arterial progenitors. A-C, Immunostain for activated (di-phosphorylated) ERK in 20-ss zebrafish embryos. Arrowhead, activated ERK within the developing vasculature. In B, dorsal side is to the right, and in C and in all other cross sections, dorsal is to the top. NC, notochord, which lies dorsal to the angioblasts. D-F, Cross sections of immunostain for activated ERK in 12-ss (15-hpf) embryo (D,E) and in 24-hpf embryo (F). E, Merged view of angioblasts, marked by GFP (green) expressed under the fli-1 vascular-specific promoter (Lawson et al., Developmental Biology 248:307-318, 2002), and activated ERK (yellow). At around 12-ss, activated ERK (yellow) is preferentially detectable in angioblasts that reach the midline earlier than the rest of angioblasts (green). F, By around 24-hpf, activated ERK is no longer detected in angioblasts. G-1, Cross section of 20-ss embryo. G, Angioblasts marked by vascular-specific GFP (green). H, Immunostain for activated ERK (red). J, Merged view of activated ERK and GFP, with overlap in yellow. At this stage, detectable ERK activation is restricted to the dorsal-most angioblasts (yellow). J, M, Quantitative Western analysis of ERK phosphorylation in 20-ss embryos. Error bars, standard error. J, Inhibition of ERK phosphorylation by cyclopamine (50 μM) or 676475 (25 μg/mL) treatment, starting at 10-hpf. Results normalized from 3 independent experiments. P=0.03 for cyclopamine; P<0.0001 for 676475, versus untreated. M, Enhancement of ERK phosphorylation by GS4898 (10 μM) or LY294002 (15 μM) treatment, starting at 10-hpf. Results normalized from 7 independent experiments. P=0.04 for GS4898; P=0.009 for LY294002, versus untreated. K, L, Merged view of activated ERK and GFP in embryos treated with cyclopamine (K) or 676475 (L). Note the loss of ERK activation with cyclopamine and 676475 treatments. N, O, Merged view of activated ERK and GFP in embryos treated with GS4898 (N) and LY294002 (O) showing the relative expansion in the overlap (yellow) between angioblasts and activated ERK.

FIG. 3. Diphosphorylated ERK is a critical determinant of the arterial fate. A, 30-hpf embryo. Red line, dorsal aorta (DA). Blue line, posterior cardinal vein (PCV), which terminates at the common cardinal vein (CCV). Yellow box, region shown in B. Green vertical line, cross sections in C-F. B, In situ hybridization of 30-hpf embryos with the arterial marker ephrin-B2a. Left, normal ephrin-B2a expression (arrowhead) in untreated embryos. Middle, intact ephrin-B2a expression in embryo treated with high dose (15 μM) GS4898. Right, ephrin-B2a expression is lost in SL327-treated (60 μM) embryos. C, Cross sections of embryos shown in B. Left, in untreated embryos, ephrin-B2a is expressed in the DA (black arrowhead), but not in the PCV (white arrowhead). Middle, in GS4898-treated embryos, the ephrin-B2a expressing DA is prominent, but the PCV is not visible. Right, in SL327-treated embryos, neither ephrin-B2a expression nor the DA is observed. D-F, Cross sections of 48-hpf embryos immunostained for GFP (brown) expressed in endothelial cells under the vascular-specific fli-1 promoter. Arrowheads indicate the aorta. D, In untreated embryo, both the dorsal aorta (A) and the posterior cardinal vein (V) are prominent. E, In embryo treated with 15 μM GS4898, duplication of the aortae is observed. F, In embryo treated with 100 μM U0126, the aorta is greatly reduced, whereas the vein (V) is enlarged. G, Top, fluorescent images of 48-hpf embryos expressing GFP in endothelial cells. Dorsal view of the torso at the DA bifurcation and the PCV terminating at the CCV. Head is above, and tail is below. Bottom, cartoon representations of the fluorescent images. Left, in untreated embryos, the CCV, via which blood from the tail drains to the heart, is situated lateral to the DA, which delivers blood to the tail. At the levels of the lower trunk and tail, the DA is situated dorsal to the PCV. Middle, in GS4898-treated embryos, the DA is partially duplicated, and the CCV, situated lateral to the DA, is greatly reduced. Right, in SL327-treated embryos, the DA is missing and the CCV is significantly enlarged.

FIG. 4. Genetic manipulation of AKT activity impacts artery-vein specification. A-C, Overlay of GFP-fluorescence image onto bright field image of a live 48-hp embryo injected with either pAdTrackCMV.AA-AKT (dominant negative AKT), pAdTrackCMV.myr-AKT (constitutively active AKT), or pAdTrackCMV (GFP alone). A, Tail region of a plasmid-injected embryo expressing GFP in small subsets of cells. Yellow box, region represented in B and C. B, High magnification overlay image of a typical GFP+ patch scored as arterial (red asterisk). It overlaps with the aorta (A), with a robust arterial flow (red arrow). C, Overlay image of a typical GFP+ patch scored as venous (blue asterisk). It overlaps with the caudal vein, with a robust venous flow. D, Numbers of GFP+ patch in the aorta and in the caudal vein in embryos injected with pAdTrackCMV.AA-AKT (DN-AKT), pAdTrackCMV.myr-AKT (myr-AKT), and with pAdTrackCMV (GFP only). A significant skewing of vascular GFP+ cells in favor of aorta is noted in DN-AKT injected embryos (P=0.0283, versus GFP alone), while a significant skewing of vascular GFP+ cells in favor of vein is noted in myr-AKT injected embryos (P=0.0039, versus GFP alone). Total of over 200 embryos were injected with each expression construct, and results were scored by an observer blinded to the injected construct.

FIG. 5. Limiting the compound exposure to critical times in vasculogenesis allows a relatively targeted perturbation of artery-vein specification, while minimizing gross embryonic defects or lethality. A, 36-hpf uninjected embryo. B, 36-hfp embryo treated with 10 μM GS4898 starting at 12-hpf. C, 36-hpf embryo treated with 60 μM SL327 starting at 12-hpf. Treatment with either compound starting at 12-hpf causes a moderately deformed tail curvature, but otherwise grossly intact head organogenesis, somitogenesis, and yolk extension. Apart from perturbed artery-vein identity of the axial vasculature, treated embryos are not readily distinguishable from one another. Treatment of embryos at earlier stages results in high degree of embryonic lethality and variable gross morphologic defects.

### DETAILED DESCRIPTION

Molecular and genetic studies in zebrafish have highlighted the key roles of sonic hedgehog (shh), vascular endothelial growth factor (VEGF), notch, and gridlock (grl) in the signaling pathway for vertebrate arterial development. The zebrafish is readily amenable to chemical genetic analysis, in which small molecules discovered by phenotype-based screens are used as tools for dissecting developmental processes. Using this approach, we have discovered that PI3K (PI3K) inhibitors, such as LY294002 and wortmannin, can suppress the gridlock vascular defect. We have also identified a gridlock suppressor, GS4898, a flavone that also acts as a PI3K inhibitor and inhibits activation of AKT, a downstream mediator of PI3K signaling. Further, we have found that expression of a dominant-negative form of AKT promotes arterial specification.

In developing embryos, inhibition of PI3K, a major component of VEGF signaling, results in increased activation of a distinct VEGF signaling component, the p44 Mitogen-Activated Protein (MAP) kinase/Extracellular signal-Regulated Kinase (ERK) (hereinafter referred to as “ERK”). Interestingly, ERK activation is normally restricted to the dorsal-most angioblasts, which give rise to the dorsal aorta. Inhibition of arterial differentiation by blocking shh or VEGF signaling eliminates ERK activation in angioblasts, suggesting a role for ERK activation in arterial specification. Together, our results support a model in which the midline signals shh and VEGF induce arterial differentiation by activating ERK specifically in adjacent dorsal-most angioblasts. Furthermore, chemical suppression of impaired arteriogenesis by PI3K inhibitors, such as GS4898, LY294002, and wortmannin, lifts the inhibitory effects of PI3K signaling on ERK activation.

The invention thus provides methods of treating or preventing vascular disease or promoting arterial vascular growth or development. According to the methods of the invention, arterial vascular growth or development can be promoted by inhibition of PI3K and/or AKT, or by activation of ERK pathway members (e.g., PLC-γ, PKC, Raf, MEK, and/or ERK). These methods can be useful, for example, in the treatment of diseases or conditions associated with ischemia or immature blood vessel growth. The invention also features methods for inducing angioblasts to undergo arterial differentiation by inhibiting PI3K and/or AKT, or by activating ERK pathway members (e.g., PLC-γ, PKC, Raf, MEK, and/or ERK).

As is discussed further below, ERK pathway activation can be achieved by use of an agent (e.g., a small organic molecule) that inhibits PI3K and/or AKT, or an agent that activates ERK (and/or an ERK pathway component). Alternatively, ERK pathway activation can be achieved by use of gene therapy approaches to express ERK pathway components, or by use of antisense, siRNA, and/or dominant-negative approaches to block PI3K and/or AKT.

The invention also provides methods for promoting venous vascular growth or development, as well as methods of inducing angioblasts to undergo venous differentiation, which involve activation of PI3K and/or AKT, or inhibition of ERK pathway members (see above). These methods can involve the use of agents that have such activating or inhibiting effects, or by gene, antisense, siRNA, or dominant-negative therapy approaches, as described further below.

The invention further provides pharmaceutical compositions that can be used in the methods of the invention. For example, the invention includes compositions that include one or more activators of ERK (e.g., one or more PI3K inhibitors), optionally, in combination with one or more additional agents, such as a VEGF activator (e.g., GS4012 or a derivative thereof; see, e.g., WO 2005/007159 A1). In other examples, compositions of the invention include agents used in the therapeutic methods mentioned above. The methods and compositions of the invention are described further, as follows.

### Therapy

Conditions Associated with Ischemia

The methods and pharmaceutical compositions of the invention can be used to promote arterial vascular growth for the treatment or prevention of conditions associated with ischemia. Such conditions include, for example, stroke or heart attack. In addition, the methods and compositions can be used to accelerate wound healing, promote vascularization of surgically transplanted tissue, and enhance the healing of a surgically-created anastomosis.

A patient can be suffering from or be at risk of suffering from ischemic damage when one or more tissues within the patient are deprived of an adequate supply of oxygenated blood. The interruption of the supply of oxygenated blood is often caused by a vascular occlusion, which can be caused by, for example, arteriosclerosis, trauma, or surgical procedures. There are many ways to determine if a tissue is at risk of suffering ischemic damage from an undesirable vascular occlusion. For example, in myocardial disease, these methods can include a variety of imaging techniques (e.g., radiotracer methodologies, such as ⁹⁹ mTc-sestamibi, x-ray, and MRI angiography) and physiological tests. The induction of vascular growth in a tissue affected by or at risk of being affected by a vascular occlusion, using the methods and compositions of the invention, can be an effective means for preventing and/or attenuating ischemia in such a tissue.

In ischemic conditions, the blood supply to discrete organs, such as the brain, heart, pancreas, or limbs, can be attenuated by disease, trauma, surgery, or other events. The alleviation of such attenuated blood supply, regardless of its origin, is included in the invention. Thus, prevention or alleviation of damage from indications such as myocardial ischemia and stroke are included. Additionally, the planning of a surgical procedure can be predictive of the interruption of blood supply through a particular portion of a patient's vasculature. Prior or concurrent treatment according to the invention can substantially improve the outcome of these surgeries.

### Age Related Macular Degeneration

The methods and compositions of the invention can be used to treat the wet/exudative form of age related macular degeneration (AMD), a leading cause of vision loss. The hallmark pathophysiology of AMD is abnormal growth (angiogenesis) of blood vessels, which frequently rupture or leak, leading to damage of the macula. The methods of the invention can be used to block the PI3-kinase arm of the VEGF signaling, thereby augmenting arteriogenesis by preferentially inducing artery differentiation in endothelial precursor cells in vivo. Using this approach it can be possible to stabilize vascular integrity (and hence prevent damage-inducing vascular leakage and rupture) by triggering an arterial differentiation program in the neovasculature, without eliminating completely the compensatory (and potentially beneficial) effects of angiogenesis.

### Diabetic Retinopathy

The methods and compositions of the invention can also be used to treat diabetic retinopathy. As in AMD, an important hallmark of the pathophysiology of diabetic retinopathy is abnormal growth (angiogenesis) of blood vessels, which frequently rupture or leak, leading to damage of the macula. In diabetic retinopathy, the initial angiogenic trigger may be small artery vasculopathy leading to chronic ischemia. The underlying pathophysiology is occlusive vasculopathy. Using the methods and compositions of the invention, diabetic retinopathy can be treating by selectively blocking one component of VEGF signaling, rather than eliminating completely the compensatory (and potentially beneficial) effects of VEGF-induced angiogenesis.

### Tissue and Cell Engineering

The methods and compositions of the invention can be used to engineer vascular tissues in vitro or ex vivo, for use as replacement vessels. In general, arterial conduits are preferred over venous, as they are less thrombogenic and less prone to occlusion. However, the majority of conduits used in coronary artery bypass grafting (CABG) are saphenous veins. Thus, in one example of a method of the invention, ERK activators (e.g., PI3K inhibitors) can be used to induce differentiation (e.g., arterial differentiation) in saphenous veins ex vivo, which can then be used in bypass surgery. A variety of existing ex vivo vascular remodeling methods can be used in conjunction with the methods of the invention including, for example, those described in U.S. Patent Publication No. 20030097040.

In addition, agents described herein (e.g., ERK activators, such as PI3K inhibitors) and the gene therapy approaches described herein (in particular, ex vivo gene therapy approaches) can be used in the preparation of stem cells, such as endothelial stem cells, for use in therapeutic methods. Treatment of stem cells in this manner can promote arterial (or venous) differentiation of the cells, which then can be used in many applications including, for example, the treatment of saphenous veins used in bypass surgery. The cells can also be used to promote wound healing and in other therapeutic applications such as those described herein, as well as in tissue engineering approaches, as noted above.

### Disorders of the Aorta

The methods and compositions of the invention can also be used in the treatment of dysplasias of the aorta. In particular, the aorta, which is the main trunk of the systemic arterial network, is subject to several congenital and acquired disorders that can lead to severe complications in infancy and adulthood. Coarctation of the aorta is one of the most common human congenital cardiovascular diseases. In coarctation, a discrete, localized vascular malformation partially obstructs the descending aorta, the major artery to the body, and most frequently occurs distal to the origins of vessels supplying the head and arms. Its effects often become more physiologically severe at birth, when closure of the ductus can exacerbate the restriction to aortic blood flow. As a consequence of coarctation, affected individuals suffer from high blood pressure in the upper extremities and head, and from low pressure in the trunk and legs. Survival often depends on the development of collateral blood vessels, which facilitate blood circulation in a manner so as to bypass the lesion. The methods and compositions of the invention thus can be used to treat patients with coarctation of the aorta, as well as other aortic diseases, such as interrupted arch disease.

### PI3K Inhibitors

The methods of the invention focused on promoting arterial growth or development (or differentiation of angioblasts into arterial cells) can be carried out with any agent that activates ERK and/or an ERK pathway member, such as a PI3K inhibitor (and/or an AKT inhibitor). For example, the agent can be a small molecule or peptide that inhibits the activity of PI3K and/or AKT.

### Peptide Inhibitors

SH2 domains are homologous sequences of approximately 100 amino acids found in a variety of important signal transducing molecules, where they facilitate a key component of PTK mediated cellular signaling by promoting protein-protein associations (Margolis, Growth Differ. 3:73-80, 1993; Panayotou et al., Bioessays 15:171-177, 1993; and Pawson et al., Curr. Biol. 3:434-442, 1993). Among different classes of SH2 domains, a secondary ligand specificity resides within the amino acid sequence neighboring the pTyr residue, particularly in residues toward the C-terminal side, thereby allowing families of SH2 domains to “recognize” specific binding sites on target proteins. Small pTyr-bearing peptides modeled after these target sequences also bind with high affinity and moderate selectivity to the appropriate SH2 domains, thereby providing a potential means of competitively inhibiting specific SH2 signaling pathways (Fantl et al., Cell 69:413-423, 1992; Songyang et al., Cell 72:767-778, 1993).

Peptides exhibiting inhibitory potency against the PI3-kinase p85 C-terminal SH2 domain can be derived, for example, from Tyr751 of the PDGF receptor (see Piccione et al., Biochemistry 32:3197-3202, 1993), and synthesized by introducing the pTyr mimetic L-O-malonyl tyrosine (L-OMT) into the peptide using solid phase peptide techniques such as those described in U.S. Pat. No. 5,688,992 and by Ye et al., J. Med. Chem. 38:4270, 1995.

PI3K inhibitory peptides that can be used in the methods of the invention include, without limitation, Ac-Asp-Tyr(2-malonyl)-Val-Pro-Met-Leu-NH₂ trimethyl ester (referred to herein as PI3K-SH2-OMT). This peptide is commercially available from BIOMOL® International (Cat. No. ST-425).

### Small Molecule Inhibitors

Small molecule inhibitors that can be used in the methods of the invention focused on arterial differentiation include, without limitation, wortmannin, LY294002, and compounds of formula (I) (e.g., GS4898):

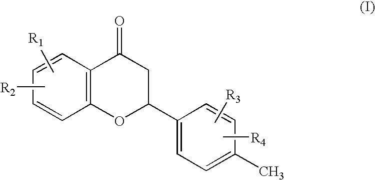

In formula (I), each of R₁, R₂, R₃, and R₄ is, independently, selected from H, halide, CF₃, C₁₋₃ alkyl (unsubstituted or substituted), C₁₋₃ alkoxy, OH, SH, NO₂, and CN.

### Synthesis of Compounds of Formula I

Compounds of formula (I) can be prepared using commercially available starting materials and established synthetic protocols. Numerous methods are available for the synthesis of compounds of formula (I). For example, compounds of formula (I) can be prepared as described in Scheme 1. See, for example, Bondag et al., Turk. J. Chem. 23:163, 1999. In Scheme 1, R₁, R₂, R₃, and R₄ are as defined above. Numerous hydroxyacetophenones and acid chlorides (i.e., for use in reaction Scheme 1) are commercially available or are readily synthesized from commercially available starting material using standard synthetic techniques. Using the approach described in Scheme 1, GS4898 can be prepared by the reaction of 2-hydroxyacetophenone (Aldrich Cat. No. H1, 860-7) with phenylacetylchloride (Aldrich Cat. No. P1, 675-3).

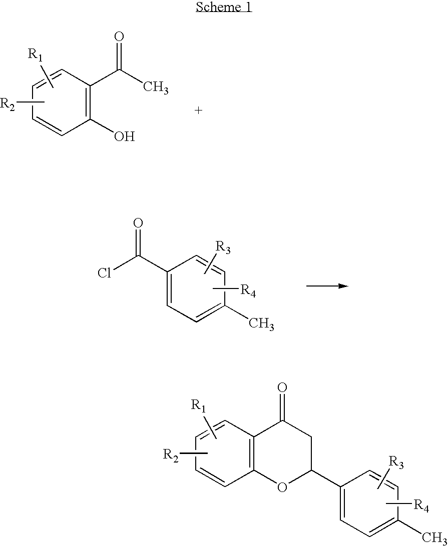

In some instances, the synthesis will require the selective protection and deprotection of alcohols, amines, sulfhydryls, and/or carboxylic acid functional groups. This can be achieved using known techniques. For example, commonly used protecting groups for amines include carbamates, such as tert-butyl, benzyl, 2,2,2-trichloroethyl, 2-trimethylsilylethyl, 9-fluorenylmethyl, allyl, and m-nitrophenyl. Other commonly used protecting groups for amines include amides, such as formamides, acetamides, trifluoroacetamides, sulfonamides, trifluoromethanesulfonyl amides, trimethylsilylethanesulfonamides, and tert-butylsulfonyl amides. Examples of commonly used protecting groups for carboxylic acids include esters, such as methyl, ethyl, tert-butyl, 9-fluorenylmethyl, 2-(trimethylsilyl)ethoxy methyl, benzyl, diphenylmethyl, O-nitrobenzyl, ortho-esters, and halo-esters. Examples of commonly used protecting groups for alcohols include ethers, such as methyl, methoxymethyl, methoxyethoxymethyl, methylthiomethyl, benzyloxymethyl, tetrahydropyranyl, ethoxyethyl, benzyl, 2-napthylmethyl, O-nitrobenzyl, P-nitrobenzyl, P-methoxybenzyl, 9-phenylxanthyl, trityl (including methoxy-trityls), and silyl ethers. Examples of commonly used protecting groups for sulfhydryls include many of the same protecting groups used for hydroxyls. In addition, sulfhydryls can be protected in a reduced form (e.g., as disulfides) or an oxidized form (e.g., as sulfonic acids, sulfonic esters, or sulfonic amides). Protecting groups can be chosen such that selective conditions (e.g., acidic conditions, basic conditions, catalysis by a nucleophile, catalysis by a lewis acid, or hydrogenation) are required to remove each, exclusive of other protecting groups in a molecule. The conditions required for the addition of protecting groups to amine, alcohol, sulfhydryl, and carboxylic acid functionalities and the conditions required for their removal are provided in detail in T. W. Green and P. G. M. Wuts, Protective Groups in Organic Synthesis (2^{nd} Ed.), John Wiley & Sons, 1991 and P. J. Kocienski, Protecting Groups, Georg Thieme Verlag, 1994 (the contents of which are incorporated herein by reference).

### Formulation and Administration

Therapeutic agents for use in the invention (e.g., PI3K inhibitors) can be administered by any appropriate route for promoting vascular growth (e.g., arterial or venous growth) or for the treatment or prevention of the diseases or conditions described elsewhere herein (e.g., those associated with ischemia). They can be administered with a pharmaceutically acceptable diluent, carrier, or excipient, in unit dosage form. Administration can be by any appropriate route including, for example, the following: topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, oral, or by suppository. In the case of ophthalmic applications, administration can be achieved by the use of topical approaches involving, for example, eyedrops and/or ophthalmic ointments, as are known in the art. In addition, intravitreous injection can also be used for ophthalmic applications.

The exemplary dosages of the agents (e.g., PI3K inhibitors) to be administered will depend on such variables as the type and extent of the disorder, the overall health status of the patient, the therapeutic index of the selected agent, and the route of administration. Standard clinical trials can be used to optimize the dose and dosing frequency for any particular pharmaceutical composition of the invention.

Therapeutic formulations can be in the form of liquid solutions or suspensions; for oral administration, formulations can be in the form of tablets or capsules; and for intranasal formulations, in the form of powders, nasal drops, or aerosols. Methods for making formulations are well known in the art and can be found, for example, in Remington: The Science and Practice of Pharmacy (20^{th} ed., ed. A. R. Gennaro), Lippincott Williams & Wilkins, 2000. Formulations for parenteral administration can contain, for example, excipients, sterile water, or saline; polyalkylene glycols such as polyethylene glycol; oils of vegetable origin; or hydrogenated napthalenes. Biocompatible, biodegradable lactide polymer, lactide/glycolide copolymer, or polyoxyethylene-polyoxypropylene copolymers can be used to control the release of the compounds. Nanoparticulate formulations (e.g., biodegradable nanoparticles, solid lipid nanoparticles, liposomes) can be used to control the biodistribution of the compounds. Other parenteral delivery systems that can be used in the invention include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycholate and deoxycholate, or may be oily solutions for administration in the form of nasal drops, or as a gel. The concentration of the compound in the formulation will vary depending upon a number of factors, including the dosage of the drug to be administered, and the route of administration.

Therapeutic agents of the invention (e.g., PI3K inhibitors) can optionally be administered as pharmaceutically acceptable salts, such as a non-toxic acid addition salt or a metal complex that is commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acids such as hydrochloric acid, hydrobromic acid, sulfuric acid, phosphoric acid, or the like. Metal complexes include zinc, iron, and the like.

Controlled release formulations are useful for agents that have (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; or (iii) a short biological half-life, so that frequent dosing during a day is required in order to sustain the plasma level at a therapeutic level.

Many strategies can be pursued to obtain controlled release in which the rate of release outweighs the rate of metabolism of the agent. For example, controlled release can be obtained by the appropriate selection of formulation parameters and ingredients (e.g., appropriate controlled release compositions and coatings). Examples include single or multiple unit tablet or capsule compositions, oil solutions, suspensions, emulsions, microcapsules, microspheres, nanoparticles, patches, and liposomes.

Formulations for oral use include tablets containing the active ingredient(s) in a mixture with non-toxic pharmaceutically acceptable excipients. These excipients can be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

Formulations for oral use can also be provided as chewable tablets, or as hard gelatin capsules in which the active ingredient is mixed with an inert solid diluent, or as soft gelatin capsules in which the active ingredient is mixed with water or an oil medium.

In addition to being administered to patients in therapeutic methods, the compounds described herein can also be used to treat cells and tissues ex vivo. For example, the compounds can be used to treat explanted tissues (e.g., saphenous veins) that may be used, for example, in transplantation.

### Gene Therapy

As is noted above, gene therapy methods are included in the invention, which can be used to promote vascular (arterial or vascular) growth or development, or for the promotion of arterial or venous development of angioblasts. As discussed above, according to the methods of the invention relating to promoting arterial growth or development, such gene therapy approaches can involve expressing ERK pathway components (e.g., phospholipase C gamma (PLC-γ), protein kinase C (PKC), Raf, MEK, and/or ERK), or by use of antisense and/or siRNA approaches to block PI3K and/or AKT. Gene therapy approaches pertaining to promoting venous growth can involve expressing PI3K and/or AKT, or by use of antisense and/or siRNA approaches to block ERK pathway components (e.g., PLC-γ, PKC, Raf, MEK, and/or ERK).

Genes encoding the proteins noted above can be introduced into angioblasts ex vivo to generate cells that can be used in cell-based therapies. The genes can also be introduced into other cells (e.g., fibroblasts) using ex vivo gene therapy approaches, and the resulting cells implanted into subjects in which the implanted cells produce the proteins. The cells used in these ex vivo gene therapy approaches can be, when possible, obtained from the subject to whom the genetically modified cells are later administered or a matched donor. Gene therapy approaches can also be used to introduce the genes (e.g., genes included in vectors) into cells in vivo. Numerous methods for gene therapy are well known in the art and can be used in the invention. These approaches can employ vectors such as viral vectors (DNA or RNA) and plasmid vectors, and/or chemical means. Examples of different approaches that can be used in the gene therapy methods of the invention are described as follows.

One example of a viral vector-based gene therapy approach that can be used in the invention employs adeno-associated virus (AAV) vectors, which can achieve latent infection of a broad range of cell types, resulting in persistent expression of a therapeutic gene in a subject. As examples, the following AAV vectors can be used: AAV1, AAV2, AAV3, AAV4, AAV5, and AAV6 (Lee et al., Biochem. J. 387:1, 2005). The capsid protein of these vectors can, optionally, be genetically modified, if desired, to direct infection towards a particular tissue type (Lieber, Nature Biotechnology 21:1011, 2003).

Other examples of virus vector-based approaches employ adenoviruses, which infect a wide variety of cell types, including non-dividing cells. The invention includes the use of any one of more than 50 serotypes of adenoviruses that are known in the art, including the most commonly used serotypes for gene therapy: type 2 and type 5. To increase the efficacy of gene expression and prevent unintended spread of the virus, adenoviruses can include genetic modifications, such as E1 region deletions, E1 region and E2 and/or E4 region deletions, or deletion of the entire adenovirus genome except for the cis-acting inverted terminal repeats and a packaging signal (Gardlik et al., Med. Sci. Monit. 11:RA110, 2005). Any adenoviral vectors including such modifications can be used in the invention.

The invention also includes the use of retroviral vectors including, for example, Moloney Murine Leukemia Virus (MoMLV). These vectors can include genetic modifications including, e.g., deletions of the gag, pol, and/or env genes, as is known in the art. Using retrovirus constructs, gene therapy vectors can be targeted to specific tissues or cells. This can be achieved by the fusion of part of the retrovirus env gene to a sequence encoding the ligand for a tissue-specific receptor. A specific type of retrovirus vector that can be used in the invention is lentivirus vectors, which can infect both proliferating and quiescent cells. An exemplary lentivirus vector for use in gene therapy is HIV-1. Previously constructed genetic modifications of lentiviruses, which can be used in vectors of the present invention, include deletions of all protein encoding genes except those encoding gag, pol, and rev (Moreau-Gaudry et al., Blood 98:2664, 2001).

In addition to the viral vectors described above, other viral vectors that can be used in the invention include, for example, vaccinia virus, bovine papilloma virus, and herpes virus, such as Epstein-Barr Virus vectors. (Also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77 S-83S, 1995).

Gene therapy methods employing chemical means for introducing nucleic acid molecules into cells can also be used in the invention. In one example, cationic liposomes are used. Exemplary cationic liposomes for use in the invention include DOTMA, DOPE, DOSPA, DOTAP, DC-Chol, Lipid GL-67™, and EDMPC. These chemicals can be used individually or in combination to transfect cells with a vector, such as a plasmid, that has been constructed to express a gene of interest. Other approaches that can be used in the invention involve the use of lipofection (Felgner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983) or asialoorosomucoid-polylysine conjugation (Wu et al., J. Biol. Chem. 263:14621, 1988; Wu et al., J. Biol. Chem. 264:16985, 1989).

In other methods, DNA-polymer conjugates can be used to express a protein of interest in a patient. In such approaches, a vector constructed to express the protein is combined with a polymer to achieve expression of protein without the use of a viral vector. Exemplary compounds for use in this approach are polyethyleneimine (PEI), polylysine, polylysine linked to nuclear localization signals, polyamidoamine, and polyarginine (Arg₁₆). Another method of gene therapy that can be used in the invention employs a substantially purified DNA vector (naked DNA) for the expression of a therapeutic protein in a subject. Such a DNA vector can be administered by injection, use of a gene gun, or electroporation.

In the chemical-based, non-viral approaches described above, the therapeutic material can be directed to certain tissue types. For example, the material can include antibodies, such as multivalent antibodies, receptor ligands, or carbohydrates that direct the materials to the desired tissue.

In the case of ex vivo gene therapy, the vectors described above can be administered directly to cells in culture (e.g., stem cells, such as hematopoietic stem cells, hemangioblasts, or fibroblasts), which can be obtained from the patient or from a donor. In addition to such vector-based methods, gene transfer into such cells can be achieved non-vector-based methods such as those described above, as well as transfection methods involving the use of calcium phosphate, DEAE dextran, electroporation, or protoplast fusion.

In general, ex vivo gene therapy results in expression of a therapeutic gene only in a particular, desired tissue. In such applications, as well as applications in which tissue specific expression of the protein is not a concern, the vectors described above can be constructed so as to constitutively express the protein. Numerous constitutive regulatory elements that can be used in such constructs are well known in the art. For example, certain elements present in the native viruses described above can be used to constitutively express a gene of interest. Other examples of constitutive regulatory elements that can be used in the invention include β-actin, EF1, EGR1, eIF4A1, FerH, FerL, GAPDH, GRP78, GRP94, HSP70, beta-Kin, ROSA, and ubiquitin B promoters. For in vivo applications, the vectors described above can, if desired, be modified to include regulatory elements that confine the expression of the protein to certain tissue types, such as endothelial or hematopoietic cells. Numerous examples of regulatory elements specific to certain tissue types are well known in the art.

In addition to constitutive and cell/tissue-specific promoters, the gene therapy methods of the invention can employ inducible promoters. In one example of such an approach, cells are transfected with multiple viral or plasmid vectors. Typically a first vector expresses a gene of interest under the control of a regulatory element that is responsive to the expression product of a second vector. The activity of this expression product is controlled by the addition or administration of a pharmacological compound or other exogenous stimulation. Examples of these systems are those including the following inducing agents or conditions: tetracycline, mifepristone, ponasterone A, papamycin, tamoxifen, radiation, and heat shock (Robson et al., J. Biomed. Biotechnol. 2:110, 2003).

### Protein Therapy

The invention also includes therapeutic methods involving administration of proteins such as those noted above (e.g., PLC-γ, PKC, Raf, MEK, ERK, PI3K, AKT, or dominant-negative forms thereof) and/or fragments or fusions thereof. In these methods, the protein is administered alone or is conjugated to another agent that stabilizes and/or directs localization of the protein. For example, the protein can be conjugated to an agent that facilitates translocation of protein across cell membranes. In one example, protein therapy can involve the use of fusion constructs including a protein of interest and a protein transduction domain (PTD). The three most commonly used PTDs, which can be used in the invention, are from the Drosophila homeotic transcription protein antennapedia (Antp), the herpes simplex virus structural protein VP22, and the human immunodeficiency virus 1 (HIV-11) transcriptional activator Tat protein (Wadia et al., Curr. Opin. Biotechnol. 13:52, 2002). Also included in the invention is the use of modified PTDs that have enhanced translocation properties (see, e.g., Ho et al., Cancer Res., 61:474, 2001).

Under certain circumstances, it may be desirable to treat patients with a protein according to the invention through both protein and gene-based therapies. For example, gene therapy resulting in integration into host cells can result in prolonged expression, but it may take some time before therapeutic levels are reached using this approach alone. To achieve therapeutically effective concentrations of the protein shortly after the initiation of such treatment, before the gene therapy approach results in therapeutic levels, it may be desirable to carry out protein therapy. Using such an approach, therapeutic levels of the protein are obtained both in the short and long term. In other circumstances, only short-term therapy may be desired. In such cases, it may be beneficial to carry out protein therapy alone (i.e., in the absence of gene therapy). In addition to these combinations, protein and/or nucleic acid molecule-based therapies can also be combined with any other type of therapy, such as those described elsewhere herein (e.g., small molecule therapy).

As is discussed elsewhere herein, standard methods can be used to administer proteins according to the methods of invention. Such approaches include, for example, subcutaneous and intramuscular injection. In other examples, systemic (e.g., intravenous or oral) is used. Other modes of administration, including others described herein, can be selected by those of skill in the art.

### Interferring RNA

The invention also includes the use of interfering RNA (RNAi) in methods to disrupt expression of genes noted above (e.g., genes encoding PLC-γ, PKC, Raf, MEK, ERK, PI3K, or AKT). The term “RNAi” is used herein to refer collectively to several gene silencing techniques, including the use of siRNA (short interfering RNAs), shRNA (short hairpin RNA—an RNA bearing a fold-back stem-loop structure), dsRNA (double-stranded RNA; see, for example, Williams, Biochem. Soc. Trans. 25:509, 1997; Gil and Esteban, Apoptosis 5:107, 2000; Clarke and Mathews, RNA 1:7, 1995; Baglioni and Nilsen, Interferon 5:23, 1983), miRNA (micro RNAs), stRNAs (short (or “small”) temporal RNAs), and the like, all of which can be used in the methods of the present invention.

As is known in the art, such approaches can include the use of sense and/or antisense sequences or regions that are generally covalently linked by nucleotide or non-nucleotide linker molecules, as is known in the art. Alternatively, the linkages can be non-covalent, involving, for example, ionic, hydrogen bonding, Van der Waals, hydrophobic, and/or stacking interactions. siRNAs of the invention can be, e.g., between 19 and 29 nucleotides in length, while dsRNAs can be at least 30, 50, 100, or 500 nucleotides in length. As is known in the art, shRNAs are generally designed to form double-stranded regions of 19 to 29 nucleotides in length, although these lengths can vary (see Paddison et al., Genes Dev. 16:948, 2002). Exemplary requirements for siRNA length, structure, chemical composition, cleavage site position, and sequences essential to mediate efficient RNAi activity are described, for example, by Elbashir et al., EMBO J. 20:6877, 2001; and Nykanen et al., Cell 107:309, 2001.

RNAi molecules of the present invention include any form of RNA, such as partially purified RNA, essentially pure RNA, synthetic RNA, recombinantly produced RNA, as well as RNA that differs from naturally occurring RNA by the addition, deletion, substitution, and/or alteration of one or more nucleotides. Such alterations can include the addition of non-nucleotide material to, e.g., the end(s) of the RNA or internally (at one or more nucleotides of the RNA), or the RNA molecule can contain a 3′hydroxyl group. Nucleotides in the RNAi molecules of the present invention can also include non-standard nucleotides, including non-naturally occurring nucleotides or deoxyribonucleotides. Examples of modified nucleotides that can be included in RNAi molecules of the invention, such as 2′-O-methyl ribonucleotides, 2′-deoxy-2′-fluoro ribonucleotides, “universal base” nucleotides, 5-C-methyl nucleotides, nucleotides with phosphorothioate internucleotide linkages, and inverted deoxyabasic residues, are described, for example, in U.S. Patent Application Publication No. 20040019001. RNAi molecules can be used individually, or in combination with other RNAi constructs.

The invention is based, in part, on the experimental results described below.

### Experimental Results

Angioblasts are multipotent progenitor cells that can give rise to arteries or veins (Lawson et al., Nature Reviews Genetics 3:674-682, 2002). Genetic disruption of the gridlock gene is known to perturb the artery-vein balance resulting in generation of insufficient numbers of arterial cells (Zhong et al., Nature 414:216-220, 2001). However, the precise biochemical signals within angioblasts that determine the artery-vein cell fate decision are poorly understood. We have identified by chemical screening two classes of compounds that compensate for a mutation in the gridlock gene (Peterson et al., Nature Biotechnology 22:595-599, 2004). Both target the VEGF signaling pathway and reveal two downstream branches emanating from the VEGF receptor with opposing effects on arterial specification. We show that the activation of ERK (p42/44 MAP kinase) is a specific marker of early arterial progenitors and is among the earliest known determinants of arterial specification. In the early embryo, cells fated to contribute to arteries express high levels of activated ERK, whereas cells fated to contribute to veins do not. Inhibiting the phosphatidylinositol-3 kinase (PI3-K) branch with GS4898 or known PI3-K inhibitors, or with mosaic expression of dominant negative form of AKT, a downstream mediator of PI3-K, promotes arterial specification. Conversely, inhibition of the ERK branch blocks arterial specification, and mosaic expression of constitutively active form of AKT promotes venous specification. In summary, chemical genetic analysis has uncovered unanticipated opposing roles of PI3-K and ERK in artery-vein specification.

### Results and Discussion

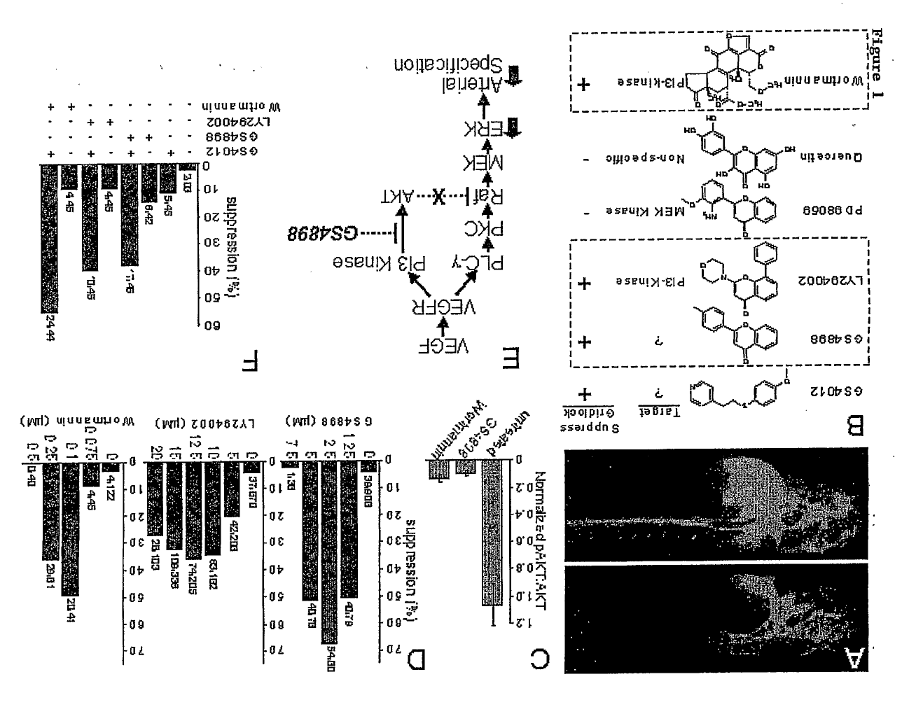

How arterial and venous cells arise from common angioblast progenitors to form a functional vasculature poses a challenging biological question. Artery-versus-vein (A-V) specification is established prior to the onset of circulation (Zhong et al., Nature 414:216-220, 2001; Jain, Nature Medicine 9:685-693, 2003; Zhong, Curr. Top. Dev. Biol. 71:53-81, 2005), and once specified, arterial and venous progenitors migrate to the appropriate locales and coalesce into functional vessels. The transcriptional repressor hey2, encoded by the gridlock gene in zebrafish, is an important determinant of the arterial fate, as evidenced by the fact that disruption of gridlock results in formation of insufficient numbers of arterial cells that leads to reduction or loss of the aorta (Zhong et al., Nature 414:216-220, 2001; Zhong, Science 287:1820-1824, 2000). Consequently, zebrafish with a mutation (grl^{m145}) in hey2/gridlock lack trunk and tail circulation because of an atresia in the proximal aorta resembling congenital aortic coarctation in humans (FIG. 1A) (Weinstein et al., Nature Medicine 1:1143-1147, 1995). In the mouse, defects in the hey2 gene contribute to vascular deformities as well (Sakata et al., J. Mol. Cell. Cardiol., 2005; Fischer et al., Genes Dev. 18:901-911, 2004). Beyond gridlock, other genetic and biochemical factors are likely involved in A-V specification (Lawson et al., Development 128:3675-3683, 2001). Identifying additional factors using traditional genetic approaches may be difficult, particularly if such factors are necessary for vital biological processes that occur prior to formation of the vasculature. To identify novel factors that govern the artery-vein cell fate decision, it will be important to employ a strategy that enables control over the dose and timing of gene inactivation. Therefore, instead of traditional genetic approaches, we have employed small molecule screening to identify conditional modifiers of the artery-vein cell fate decision.

Previously, chemical screening was performed with whole zebrafish embryos, and a small molecule, GS4012, was identified that suppresses the vascular defect in gridlock mutant embryos (Peterson et al., Nature Biotechnology 22: 595-599, 2004). GS4012 was postulated to function via activation of the VEGF signaling pathway. We have used a similar screening approach to identify a distinct compound class that is also capable of suppressing the gridlock phenotype (FIG. 1A). These compounds, represented by the compound GS4898, possess a mechanism of action that is distinct from that of GS4012. We have used these two classes of gridlock suppressors to reveal the biochemical basis for artery-vein specification during embryogenesis.

GS4898, which is structurally distinct from GS4012 (FIG. 1B), was identified in a screen of 7000 uncharacterized small molecules based on its ability to restore trunk and tail circulation to zebrafish embryo that are homozygous gridlock (grl^{m145}) mutants. GS4898 is a flavone structurally similar to several small molecule inhibitors of protein kinases (Cohen, Nature Reviews Drug Discovery 1:309-315, 2002; Walker et al., Molecular Cell 6:909-919, 2000), which suggested that GS4898 might function by targeting a kinase. We tested structurally related flavone kinase inhibitors and found that LY294002, a specific phosphatidylinositol-3 kinase (PI3-K) inhibitor is also capable of suppressing the gridlock phenotype (FIG. 1B). In addition, the structurally unrelated PI3-K inhibitor wortmannin suppresses the gridlock phenotype. Moreover, GS4898 inhibits activation of AKT, a downstream mediator of PI3-K signaling (Zachary et al., Cardiovascular Research 49:568-581, 2001), in whole zebrafish embryos (FIG. 1C). These data strongly suggest that PI3-K inhibition underlies the suppression of the gridlock phenotype by GS4898.

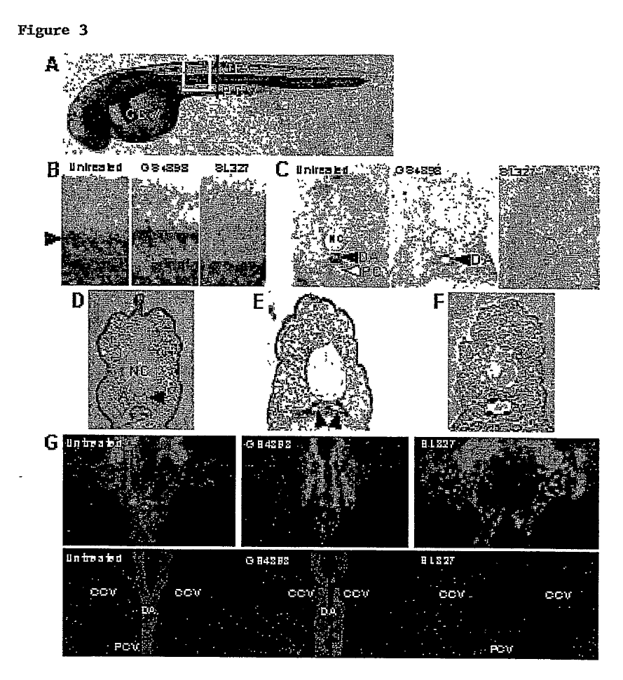

GS4898 is capable of suppressing the gridlock phenotype in the 1- to 5 μM range (FIG. 1D). At higher doses, GS4898, LY294002, and wortmannin each causes severe vascular defects (FIG. 3C). As discussed below, such high dose responses may result from severe perturbations in angioblast cell fate determination caused by a complete inhibition of PI3-K. Thus, suppression of the gridlock phenotype by GS4898 appears to require a partial inhibition of PI3-K, sufficient to overcome deficient arterial cell formation, but not sufficient to grossly distort the artery-vein balance.

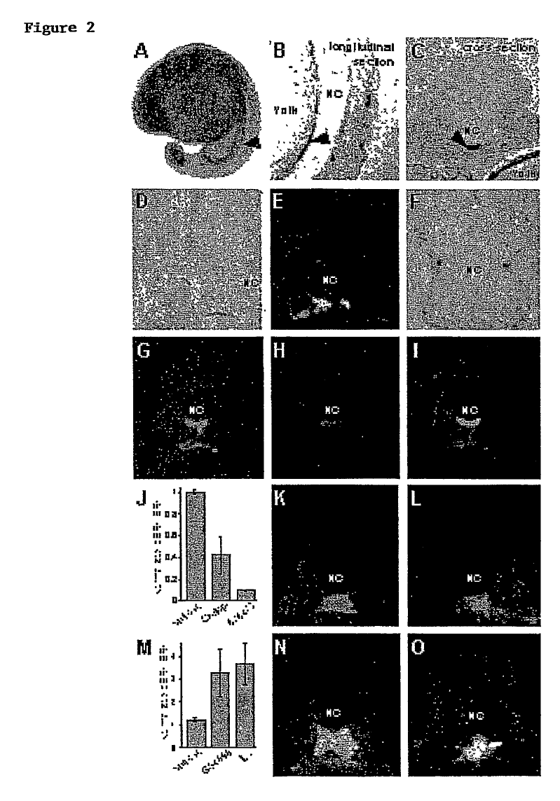

Suppression of the gridlock vascular defect by inhibition of PI3-K, a well-known mediator of VEGF signaling, is intriguing because GS4012 was postulated to act via activation of the VEGF signaling pathway and VEGF cDNA injection can suppress the gridlock phenotype (Peterson et al., Nature Biotechnology 22: 595-599, 2004). This apparent paradox led us to consider a hypothesis in which one of the two best characterized branches of VEGF signaling (Zachary et al., Cardiovascular Research 49:568-581, 2001), namely the PLC-γ/MAP kinase (ERK) pathway, triggers arterial fate specification, while the other, the PI3-K/Akt branch, exerts an inhibitory effect on the PLC-γ/ERK branch (FIG. 1E) (Blum et al., Journal of Biological Chemistry 276:33428-33434, 2001). Evidence for such crosstalk between the two signaling pathways has been observed at the level of direct phosphorylation of Raf by Akt in a human cancer cell line (Zimmermann et al., Science 286:1741-1744, 1999). This model is attractive because PLC-γ is known to be required specifically for artery, but not vein, development in the zebrafish (Lawson et al., Genes and Development 7:1346-1351, 2003). In this model, GS4898 compensates for the deficient artery formation in gridlock mutants by lifting PI3-K's inhibition of the arteriogenic PLC-γ/ERK branch (FIG. 1E). If correct, GS4898 and the PI3-K inhibitors LY294002 and wortmannin would be predicted to potentiate the effects of the putative VEGF pathway activator GS4012. Indeed, we find that GS4898, LY294002 and wortmannin synergize with GS4012 in suppressing the gridlock phenotype (FIG. 1F). Moreover, GS4898 or LY294002 treatment significantly increases activation of ERK kinase, a central downstream mediator of PLC-γ(Zachary et al., Cardiovascular Research 49:568-581, 2001), in whole embryos (FIG. 2M). These data support a hypothesis in which GS4898 promotes arterial specification by lifting PI3-K's inhibition of ERK signaling (FIG. 1E).

The possible involvement of ERK signaling in artery cell fate specification led us to consider whether it might be activated specifically in the pre-arterial subpopulation of angioblasts. Using an antibody specific for ERK activated by dual phosphorylation at Thr-202 and Tyr-204 (Zimmermann et al., Science 286:1741-1744, 1999; Vasioukhin et al., Cell 104:605-617, 2001), we found that activated ERK (dp-ERK) is preferentially localized to endothelial precursors fated to become arterial cells during a critical period in vasculogenesis (FIG. 2A-I). Starting around 15-hours post fertilization (hpf), activated ERK is detected in a subset of angioblasts that migrate from their origin in the lateral mesoderm toward the midline (FIGS. 2D,E). This pattern of activated ERK detected in the migrating angioblasts is reminiscent of the ERK activation in the migrating lateral tracheal branches in Drosophila (Gabay et al., Development 124:3535-3541, 1997). Most remarkably, ERK activation persists in the dorsal-most angioblasts, adjacent to the notochord (FIGS. 2H, I), but not in ventral angioblasts. By 17-hpf, these dorsal-most angioblasts begin to express the arterial marker ephrin-B2 as they differentiate into arterial endothelial cells of the aorta, which invariably forms dorsally in relation to the posterior cardinal vein (Jin et al., Development 132:5199-5209, 2005). By 24-hpf, activated ERK becomes undetectable in endothelial cells as the aorta matures (FIG. 2F). The exquisite patterns of ERK activation in the arterial progenitors and the newly differentiated arterial endothelial cells raise the intriguing possibility that ERK activation is an early marker and/or determinant of the arterial cell fate (Corson et al., Development 130:4527-4537, 2003).

If ERK activation is an arterial cell fate marker, disruption of arterial specification should lead to perturbations in ERK activation. In the zebrafish embryo, two extracellular signals, sonic hedgehog (shh) and VEGF, expressed in the notochord and somites respectively, are required for arterial specification (Lawson et al., Development 128:3675-3683, 2001; Lawson et al., Developmental Cell 3:127-136, 2002). We found that blocking arterial formation with the hedgehog pathway inhibitor cyclopamine or the VEGF receptor (type-1 and 2) inhibitor 676475 greatly reduces ERK phosphorylation on quantitative Western analysis of whole embryo lysates, and causes a loss of ERK activation within angioblasts on whole mount embryo immunostaining (FIG. 2J-L). Interestingly, treatment with GS4898 or LY294002 not only increases ERK activation on quantitative Western analysis, but also causes an expansion of ERK activation within the angioblast population (FIG. 2N-O). These data demonstrate that ERK activation is indeed an early marker of the arterial cell fate (Corson et al., Development 130:4527-4537, 2003). In addition, they suggest that PI3-K may function to spatially limit ERK activation to the dorsal-most angioblast.

If ERK activation within a specific subset of angioblasts is a determinant of the arterial fate, changes in ERK activation should lead to perturbations in artery-vein specification. Indeed, we find that augmenting ERK activation increases artery and decreases vein formation. For example, treatment with GS4898 leads to occasional duplication of the aorta (FIGS. 3E, G, middle), and to either loss or reduction of venous structures, such as the common cardinal vein (FIGS. 3C, F, G, middle). In contrast, blocking ERK activation decreases artery and increases vein formation. For example, treatment with either SL327 or U0126, inhibitors of MEK, an upstream activator of ERK, leads to either loss or reduction of aorta (FIGS. 3C, F, G, right), and to an expansion of the common cardinal vein (FIGS. 3F, G, right). In summary, stimulation of ERK activation shifts the artery-vein decision in favor of artery formation, while inhibition of ERK has the opposite effect. Therefore, activation of ERK in angioblasts is a key determinant of arterial specification.

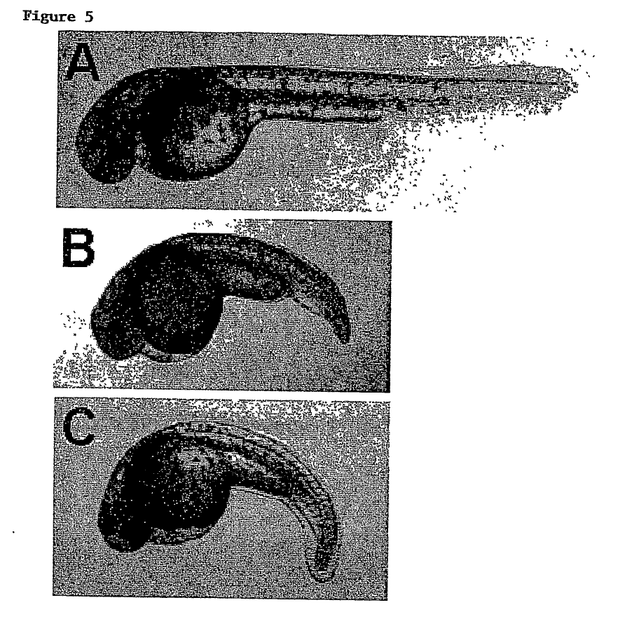

Our findings from chemical genetic approach indicate that PI3-K and ERK have surprisingly opposing roles in artery-vein specification. Because PI3-K and ERK are ubiquitously expressed signaling cassettes utilized multiple times during embryonic development, traditional genetic disruptions of these pathways result in gross perturbation of the dorsoventral axial pattern (Tsang et al., Development 131:2769-2779, 2004), and in abnormal morphogenetic movements during gastrulation (Montero et al., Current Biology 13:1279-1289, 2003). Nevertheless, mild defects such as deficient intersomitic vessel formation caused by a transient treatment with a VEGF inhibitor could be partially reversed by a low dose injection of mRNA encoding constitutively active AKT (Chan et al., Cancer Cell 1:257-267, 2002). In contrast, our attempts to overexpress constitutively active or dominant negative forms of ERK, MEK, or AKT in embryos have resulted in early embryonic deaths that preclude an adequate assessment of their effects on artery-vein specification. These results highlight an inherent advantage of the chemical genetic approach for dissection of vascular development since limiting the compound exposure to critical times in vasculogenesis allows a relatively targeted perturbation of artery-vein specification, while minimizing gross embryonic defects or lethality associated with complete disruption of a pleiotropic factor (FIG. 5).

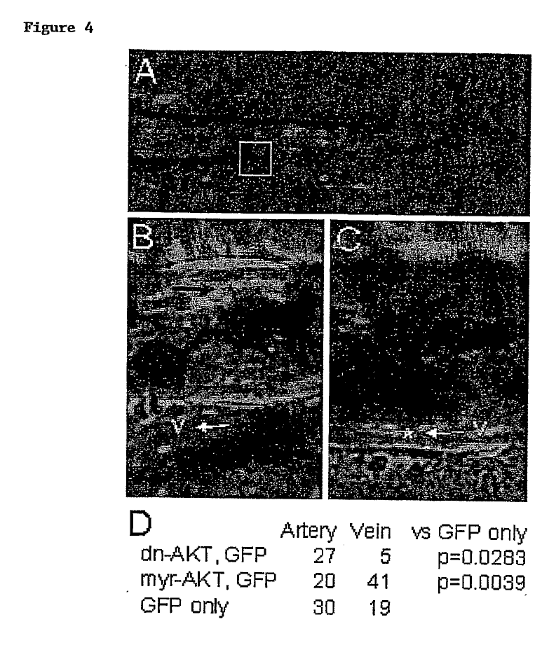

To overcome the early-lethal effects of globally perturbing PI3-K, we caused a mosaic expression of dominant negative form of AKT (DN-AKT) by injecting into 1- to 2-cell stage embryos the expression plasmid pAdTrackCMV.AA-AKT, which expresses both GFP and DN-AKT under the CMV promoter (Nagoshi et al., J. Clin. Invest. 115:2128-2138, 2005). In a similar manner, mosaic expression of constitutively active, myristoylated form of AKT (myr-AKT), was also induced along with GFP. A low dose (50-pg) injection, which permitted grossly normal development and establishment of circulation, resulted in a very small fraction (<1%) of cells that express GFP (FIG. 4A). If inhibition of the PI3-K/AKT signaling in angioblasts could drive arterial specification, the rare angioblasts that expressed the dominant negative AKT in such mosaic setting would be predicted to become arterial endothelial cells. Conversely, if activation of the PI3-K/AKT signaling in angioblast favored venous specification, angioblasts that expressed the myristoylated AKT would be predicted to become venous endothelial cells. Indeed, a blinded analysis of these GFP positive cells localizing to either the dorsal aorta (FIG. 4B) or the axial vein (FIG. 4C) indicates that GFP-positive cells were preferentially found in the dorsal aorta when injected with the construct co-expressing the dominant negative AKT (pAdTrackCMV.AA-AKT) whereas GFP-positive cells were preferentially found in the vein when injected with the construct co-expressing the myristoylated AKT (pAdTrackCMV.myr-AKT). (FIG. 4D). These results provide further support for the model in which the PI3-K/AKT signaling in angioblasts functions to block the arterial specification (FIG. 1E).

### CONCLUSIONS

The two compound classes identified by unbiased screening in mutant zebrafish have been valuable tools for studying artery-vein specification. Not only have they confirmed the importance of VEGF signaling in this process, they have revealed that two downstream components of VEGF signaling surprisingly have opposite effects on artery-vein specification of endothelial progenitor cells. ERK signaling promotes the arterial cell fate, whereas PI3-Kinase has an opposing effect by blocking ERK activation. GS4898 and known PI3-K inhibitors shift the artery-vein balance in favor of arterial development. We note with interest that suppression of the gridlock vascular defect by GS4898 likely involves a partial inhibition of PI3-K sufficient to overcome deficient arterial cell formation but not sufficient to grossly disrupt artery-vein balance. In light of this, it is doubtful that a typical loss-of-function genetic lesion in PI3-K signaling could have suppressed the gridlock phenotype. Furthermore, the use of chemical suppressors allowed focused analysis of the role of PI3-K and ERK in artery-vein specification, despite their pleiotropic requirement during early development (Tsang et al., Development 131:2769-2779, 2004; Montero et al., Current Biology 13:1279-1289, 2003). In summary, the chemical genetic approach employed here was instrumental in the discovery of previously unsuspected roles of two well-known VEGF signaling branches, demonstrating the utility and power of chemical genetics in the study of vertebrate development.

### Experimental Procedures

Screening for suppressors of the gridlock phenotype. The screen was performed as previous described with modifications (Peterson et al., Nature Biotechnology 22:595-599, 2004). Briefly, pairs of homozygous grl^{m145} zebrafish were mated, and fertilized eggs were arrayed in 96-well plates containing E3 embryo buffer. At the 6-somite stage, small molecules from the DiverSet E library (Chembridge) were added to each well, yielding a final concentration of 2.5 μM. Embryos were incubated at 28.5° C. for 48 hrs. Circulation to the tail was assessed visually using a dissecting microscope.

Fluorescent microangiography. Fluorescence microangiography was performed as previously described (Weinstein et al., Nature Medicine 1:1143-1147, 1995).

Quantitative Western Analysis. Control and treated 20-hour embryos were homogenized in SDS lysis buffer. Denatured lysates from equivalent numbers of embryos (typically 7) were loaded and separated on a 10% SDS polyacrylamide gel, and transferred to a PVDF membrane. For Akt analyses, lysates were prepared from equivalent de-yolked embryos. Membranes were blocked in 5% nonfat dry milk, and incubated with rabbit polyclonal anti-phospho-p44/42 MAP kinase (Thr202/Tyr204) antibody, anti-p44/42 MAP kinase antibody, anti-phospho-Akt (Ser472) antibody, or anti-Akt antibody (Cell Signaling Technology). The membranes were then washed, and incubated with goat anti-rabbit horseradish peroxidase (HRP)-conjugated secondary antibodies. After several washes, membranes were developed using ECL+chemiluminescence substrates and scanned using a Typhoon Imager (Amersham). Band intensities were quantified using image quantification software. After each immunoblotting, membranes were stripped according to manufacturer's instructions prior to incubation with other antibodies. For each membrane, the ratio of phospho-MAP kinase to total MAP kinase was calculated. The ratio between phospho-AKT and AKT was calculated in a similar fashion. For comparison between individual experiments, ratios were normalized to ratios obtained in control samples.

Immunostaining. Zebrafish embryos expressing EGFP under the fli-1 promoter (Lawson et al., Developmental Biology 248:307-318, 2002) were fixed in 4% paraformaldehyde, and stored in methanol at −20° C. until needed. Embryos were rehydrated, blocked in 2% sheep serum, and incubated with primary antibodies (rabbit polyclonal anti-phospho-p44/42 MAP kinase antibody, and mouse monoclonal anti-GFP antibody). After several washes in blocking solution, embryos were incubated with secondary antibodies (goat anti-rabbit antibody conjugated to either Alexa Fluor 594 or HRP, and goat anti-mouse antibody conjugated to FITC, BD Biosciences). After extensive washes, embryos were embedded in plastic (JB-4), and sectioned. Immunofluorescent images of embryo sections were then analyzed using MetaMorph image analysis software. For specimens incubated with secondary antibody conjugated to HRP, color reaction was performed prior to plastic embedment.

Whole-mount in situ labeling and histology. Whole-mount RNA in situ hybridization for ephrin-B2 expression was performed as described (Zhong et al., Nature 414:216-220, 2001). For sections, specimen were embedded in JB-4 plastic following color reaction.

cDNA injections and identification of GFP+ cells within the vasculature. Embryos were injected at the 1- to 2-cell stage with 1 nL of 50 μg/μL pAdTrackCMV.AA-AKT plasmid encoding GFP and DN-AKT, pAdTrackCMV.myr-AKT plasmid encoding GFP and myr-AKT, or pAdTrackCMV containing only GFP. Embryos were incubated at 28.5° C. for 48 hrs, and surviving embryos with grossly normal morphology and intact circulation were analyzed for presence of GFP+ cells within the vasculature using a fluorescent microscope by an observer blinded to the injected plasmid. A GFP+ cell was scored as arterial only if it lied immediately adjacent to a strong arterial stream on the same focal plane. Likewise, a GFP+ cell was scored as venous if it lied immediately adjacent to a strong venous stream on the same focal plane. Because of frequent intense fluorescence of the yolk, analysis was limited to the tail region caudal to the yolk extension.

### OTHER EMBODIMENTS

All patents, publications, and patent applications mentioned in this specification are incorporated herein by reference to the same extent as if each independent publication or patent application was specifically and individually indicated to be incorporated by reference.

While the invention has been described in connection with specific embodiments thereof, it is to be understood that it is capable of further modifications and this application is intended to cover any variations, uses, or adaptations of the invention following, in general, the principles of the invention and including such departures from the present disclosure that come within known or customary practice within the art to which the invention pertains and can be applied to the essential features hereinbefore set forth, and follows in the scope of the appended claims.

What is claimed is:

1. A method of promoting arterial vascular growth or development in a patient, the method comprising administering to the patient an agent that activates or increases the levels of p44 Mitogen-Activated Protein (MAP) kinase/Extracellular signal-Regulated Kinase (ERK) or a component of the ERK pathway in an amount sufficient to promote the vascular growth or development.

2. The method of claim 1, wherein the agent inhibits phosphatidylinositol 3-kinase (PI3K).

3. The method of claim 1, wherein the agent inhibits AKT.

4. The method of claim 1, wherein the agent is a small molecule or peptide.

5. The method of claim 4, wherein the agent is a small molecule selected from wortmannin, LY294002, and a compound of formula I: 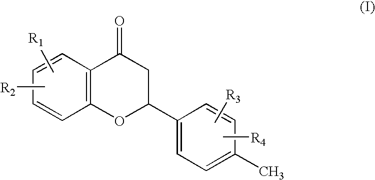
wherein each of R₁, R₂, R₃, and R₄ are, independently, selected from H, halide, CF₃, C₁₋₃ alkyl, C₁₋₃ alkoxy, OH, SH, NO₂, and CN.

6. The method of claim 5, wherein the agent is GS4898.

7. The method of claim 4, wherein the agent is an SH2 domain binding peptide.

8. The method of claim 7, wherein the peptide is PI3K-SH2-OMT.

9. The method of claim 1, wherein the agent is selected from the group consisting of PLC-γ, PKC, Raf, MEK, and/or ERK, or a nucleic acid molecule encoding PLC-γ, PKC, Raf, MEK, and/or ERK.

10. The method of claim 1, wherein the patient has or is at risk of developing ischemia.

11. The method of claim 10, wherein the ischemia is myocardial, cerebral, mesenteric, or limb ischemia, or results from a wound, surgery, vascular occlusion, or vascular stenosis.

12. The method of claim 10, wherein the patient has suffered or is at risk of suffering a heart attack or stroke.

13. The method of claim 1, wherein the patient has peripheral vascular disease.

14. The method of claim 1, wherein the arterial vascular growth or development is at the site of a wound in the patient.

15. The method of claim 1, wherein the arterial vascular growth or development is in a tissue that has been surgically implanted into said patient.

16. The method of claim 1, wherein the arterial vascular growth or development is at the site of a surgically-created anastomosis of said patient.

17. The method of claim 1, further comprising the development of a mature arterial vessel from an immature vessel.

18. The method of claim 17, wherein the immature vessel is located in the eye of the patient.

19. The method of claim 18, wherein the patient is diagnosed with diabetic retinopathy.

20. The method of claim 18, wherein the patient is diagnosed with the exudative form of age related macular degeneration.

21. A method of inducing angioblasts to undergo arterial differentiation, the method comprising contacting the angioblasts with an effective amount of an agent that activates p44 Mitogen-Activated Protein (MAP) kinase/Extracellular signal-Regulated Kinase (ERK) or a component of the ERK pathway.

22. The method of claim 21, wherein the agent inhibits phosphatidylinositol 3-kinase (PI3K).

23. The method of claim 21, wherein the agent inhibits AKT.

24. A pharmaceutical composition comprising a compound of formula I: 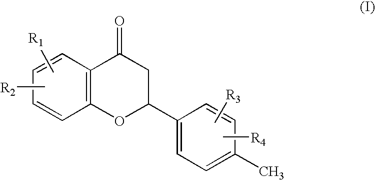
wherein,
each of R₁, R₂, R₃, and R₄ are, independently, selected from H, halide, CF₃, C₁₋₃ alkyl, C₁₋₃ alkoxy, OH, SH, NO₂, and CN;
and a pharmaceutically acceptable excipient.

25. The pharmaceutical composition of claim 24, further comprising one or more additional therapeutic agents.

26. A method of promoting venous vascular growth or development in a patient, the method comprising administering to the patient an agent that inactivates or decreases the levels of p44 Mitogen-Activated Protein (MAP) kinase/Extracellular signal-Regulated Kinase (ERK) or a component of the ERK pathway in an amount sufficient to promote the growth or development.

27. A method of inducing angioblasts to undergo venous differentiation, the method comprising contacting said angioblasts with an effective amount of an agent that deactivates or decreases levels of p44 Mitogen-Activated Protein (MAP) kinase/Extracellular signal-Regulated Kinase (ERK) or a component of the ERK pathway.
